(19) United States
(12) Patent Application Publication
(10) Pub. No.: US 2005/0272927 A1
(43) Pub. Date: Dec. 8, 2005

(54) Process for the preparation of 9, 11 epoxy steroids

(75) Inventors: Joseph J. Wieczorek, Cary, IL (US); Keith D. Maisto, Kalamazoo, MI (US); Paul David, Lindenhurst, IL (US); Marlon V. Carlos, Des Plaines, IL (US); Chung C. Kim, Northfield, IL (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia & Upjohn Company LLC, Kalamazoo, MI (US)

(21) Appl. No.: 11/086,863

(22) Filed: Mar. 22, 2005

Related U.S. Application Data
(60) Provisional application No. 60/555,418, filed on Mar. 22, 2004.

(51) Int. Cl. C07J 21/00

(52) U.S. Cl. 540/76

(57) ABSTRACT

Processes are described for epoxidation reactions. In particular, the process comprises the conversion of a steroid substrate having an olefinic unsaturation in the steroid nucleus to a structure comprising a 9,11-epoxy substituent by reaction of the substrate with a peroxide compound in the presence of a peroxide activator. The epoxidation processes described are conducted at relatively low hydrogen peroxide to steroid substrate ratio. Several optional process modifications are described.

### BACKGROUND OF THE INVENTION

This invention relates to improved processes for the preparation of 9,11 epoxy steroid compounds, especially those of the 20-spiroxane series and their analogs. More particularly, the invention is directed to novel and advantageous processes for the preparation of intermediates that can be converted to methyl hydrogen 9,11α-epoxy-17α-hydroxy-3-oxopregn-4-ene-7α,21-dicarboxylate, γ-lactone (also known as eplerenone or epoxymexrenone), or to other 9,11-epoxy, 20-spiroxane steroids.

Methods for the preparation of 20-spiroxane series compounds are described in U.S. Pat. No. 4,559,332. The compounds produced in accordance with the process of the '332 patent have an open oxygen containing ring E of the general Formula IA: 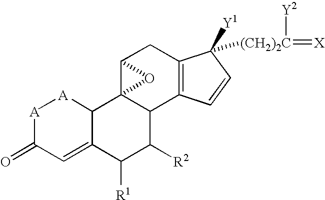
wherein Y¹, Y², -A-A-, R¹ and R² are as defined in U.S. Pat. No. 4,559,332 which is expressly incorporated herein by reference, and salts of such compounds in which X represents oxo and Y² represents hydroxy, that is to say of corresponding 17β-hydroxy-21-carboxylic acids, salts thereof, and 17-spirolactone derived therefrom.

U.S. Pat. No. 4,559,332 describes a number of methods for the preparation of epoxymexrenone and related compounds of Formula IA. The advent of new and expanded clinical uses for epoxymexrenone create a need for improved processes for the manufacture of this and other related steroids.

Novel and advantageous processes for the preparation of eplerenone are described in U.S. Pat. Nos. 6,586,591, 6,331,622, 6,180,780 and 5,981,744, each of which is expressly incorporated herein by reference.

The utility of 20-Spiroxane compounds produced in accordance with the invention is also described in Grob, U.S. Pat. No. 4,559,332.

20-Spiroxane compounds produced in accordance with the invention are distinguished by favorable biological properties and are, therefore, valuable pharmaceutical active ingredients. For example, they have a strong aldosterone-antagonistic action in that they reduce and normalize unduly high sodium retention and potassium excretion caused by aldosterone. They therefore have, as potassium-saving diuretics, an important therapeutic application, for example in the treatment of hypertension, cardiac insufficiency or cirrhosis of the liver.

20-Spiroxane derivatives having an aldosterone-antagonistic action are known, cf., for example, Fieser and Fieser: Steroids; page 708 (Reinhold Publ. Corp., New York, 1959) and British Patent Specification No. 1,041,534; also known are analogously active 17β-hydroxy-21-carboxylic acids and their salts, cf., for example, U.S. Pat. No. 3,849,404. Compounds of this kind that have hitherto been used in therapy, however, have a considerable disadvantage in that they always possess a certain sexual-specific activity which has troublesome consequences sooner or later in the customary long-term therapy. Especially undesirable are the troublesome effects that can be attributed to the anti-androgenic activity of the known anti-aldosterone preparations.

### SUMMARY OF THE INVENTION

Among the several objects of various preferred embodiments of the present invention may be noted the provision of a process for the preparation of epoxysteroid compounds; the preparation of such a process comprising oxidation of an unsaturated bond in the steroid nucleus; the provision of such process comprising epoxidation across a 9,11 double bond; and the provision of a process for the preparation of a 9,11-epoxy-20-spiroxane (i.e., 17-spirolactone) steroid such as eplerenone.

The process described herein is capable of producing eplerenone or other epoxy steroids in high yield, and allows recovery of the epoxysteroid product in high assay, and may be implemented with reasonable capital expense and conversion cost.

Briefly, therefore, the invention is directed to a process for the preparation of an epoxy steroid wherein a steroid comprising a site of unsaturation in the nucleus thereof is reacted with a peroxide compound in an epoxidation reaction zone wherein the molar charge ratio of peroxide compound to unsaturated steroid substrate is not greater than about 7.

In another aspect, the invention is directed to a process for the preparation of an epoxy steroid wherein a steroid comprising a site of unsaturation in the nucleus thereof is reacted with a peroxide compound in an epoxidation reaction zone wherein the molar charge ratio of peroxide compound to unsaturated steroid substrate and the conditions of the process are such as to avoid, or preferably entirely preclude, autocatalytic decomposition of peroxide compound.

In further aspect, the invention is directed to a process for preparing an epoxy steroid wherein the double bond carbons at the site of unsaturation are disubstituted or tri-substituted. It may be especially advantageous to employ the process of the invention in the epoxidation of a Δ^{9,11} substrate, for example, in the preparation of a 9,11-epoxy-17-spirolactone compound such as eplerenone.

The process as described herein is useful in the epoxidation of a steroid substrate in a liquid reaction medium comprising both an organic phase comprising a solvent for the steroid substrate and an aqueous peroxide solution.

As used herein, the terms “reaction mixture” and “reaction mass” are used substantially interchangeably to represent the mixture, ordinarily a two phase mixture, formed at any point in the epoxidation reaction, including the mixture obtained at the end of the reaction cycle. In certain passages, the context indicates that one or the other of these terms refers to the mixture obtained at the end of the conversion cycle.

Exemplary embodiments of the process of the present invention are further described hereinbelow and in the claims appended hereto.

Other objects and features will be in part apparent and in part pointed out hereinafter.

### DESCRIPTION OF THE PREFERRED EMBODIMENTS

Epoxidation according the process described herein may be carried out at a site of unsaturation in the steroid nucleus. As described herein, the process is especially advantageous in the epoxidation of trisubstituted bonds such as a 9,11-olefin.

Δ^{9,11}-Substrates that are useful in the process of this invention may include, for example: 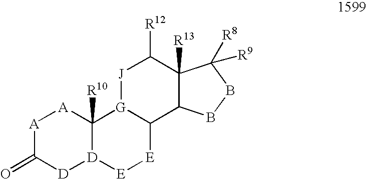
wherein

R¹⁰, R¹², and R¹³ are independently selected from the group consisting of hydrogen, halo, hydroxy, lower alkyl, lower alkoxy, hydroxyalkyl, alkoxyalkyl, hydroxycarbonyl, cyano, and aryloxy;
-A-A- represents the group —CHR¹—CHR²— or —CR¹═CR²—;
where R¹ and R² are independently selected from the group consisting of hydrogen, halo, hydroxy, alkyl, alkoxy, acyl, hydroxyalkyl, alkoxyalkyl, hydroxycarbonyl, alkoxycarbonyl, cyano, and aryloxy, or R¹ and R² together with the carbons of the steroid backbone to which they are attached form a cycloalkyl group;
-B-B- represents the group —CHR¹⁵—CHR¹⁶—, —CR¹⁵═CR¹⁶ or an α- or β-oriented group: 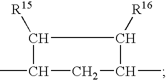
where R¹⁵ and R¹⁶ are independently selected from the group consisting of hydrogen, halo, alkyl, alkoxy, acyl, hydroxyalkyl, alkoxyalkyl, hydroxycarbonyl, alkoxycarbonyl, acyloxyalkyl, cyano, and aryloxy; or R¹⁵ and R¹⁶, together with the C-15 and C-16 carbons of the steroid nucleus to which they are attached, form a cycloalkylene group, (e.g., cyclopropylene).

R⁸ and R⁹ are independently selected from the group consisting of hydrogen, hydroxy, alkyl, alkynyl, halo, lower alkoxy, acyl, hydroxyalkyl, alkoxyalkyl, hydroxycarbonylalkyl, alkoxycarbonylalkyl, acyloxyalkyl, cyano and aryloxy, or R⁸ and R⁹ together comprise a carbocyclic or heterocyclic ring structure, or R⁸ and R⁹ together with R⁶ or R⁷ comprise a carbocyclic or heterocyclic ring structure fused to the pentacyclic D ring;
-G-J- represents the group 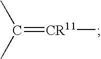
where R¹¹ is selected from the group consisting of hydrogen, alkyl, substituted alkyl and aryl;
-D-D- represents the group: 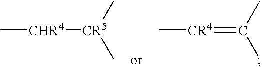
where R⁴ and R⁵ are independently selected from the group consisting of hydrogen, halo, alkyl, alkoxy, acyl, hydroxyalkyl, alkoxyalkyl, hydroxycarbonyl, alkoxycarbonyl, acyloxyalkyl, cyano and aryloxy or R⁴ and R⁵ together with the carbons of the steroid backbone to which they are attached form a cycloalkyl group;

-E-E- represents the group —CHR⁶—CHR⁷— or —CR⁶═CR⁷—;
where R⁶ is selected from the group consisting of hydrogen, halo, alkyl, alkoxy, acyl, hydroxyalkyl, alkoxyalkyl, hydroxycarbonyl, alkoxycarbonyl, acyloxyalkyl, cyano and aryloxy; and
R⁷ is selected from the group consisting of hydrogen, hydroxy, protected hydroxy, halo, alkyl, cycloalkyl, alkoxy, acyl, hydroxyalkyl, alkoxyalkyl, hydroxycarbonyl, alkoxycarbonyl, acyloxyalkyl, cyano, aryloxy, heteroaryl, heterocyclyl, acetylthio, furyl and substituted furyl, or
R⁶ and R⁷, together with the C-6 and C-7 carbons of the steroidal nucleus to which R⁶ and R⁷ are respectively attached, form a cycloalkylene group,
or R⁵ and R⁷, together with the C-5, C-6 and C-7 carbons of the steroid nucleus form a pentacyclic ring fused to the steroid nucleus and comprising a 5,7-lactol, 5,7-hemiacetal or 5,7-lactone corresponding to the structure: 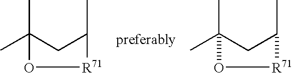
wherein R⁷¹ comprises ═CH(OH), ═CH(OR⁷²) or ═CH═O.

R¹¹ is preferably hydrogen but may also be alkyl, substituted alkyl or aryl. Where R¹¹ is substituted alkyl, substituents may include halides and other moieties which do not destabilize the epoxide ring. Where R¹′ is aryl, it may include substituents which are not strongly electron withdrawing.

In various preferred embodiments, a 3-keto structure corresponding to formula 1599, R¹², R¹⁰ and R¹³ are independently selected from the group consisting of hydrogen, fluoro, chloro, bromo, iodo, fluoromethyl, fluoroethyl, fluoropropyl, fluorobutyl, chloromethyl, chloroethyl, chloropropyl, chlorobutyl, bromomethyl, bromoethyl, bromopropyl, bromobutyl, iodomethyl, iodoethyl, iodopropyl, iodobutyl, hydroxy, methyl, ethyl, straight, branched or cyclic propyl and butyl; methoxy, ethoxy, propoxy, butoxy, hydroxymethyl, hydroxyethyl, hydroxypropyl, hydroxybutyl, methoxymethyl, methoxyethyl, methoxypropyl, methoxybutyl, ethoxymethyl, ethoxyethyl, ethoxypropyl, ethoxybutyl, propoxymethyl, propoxyethyl, propoxypropyl, propoxybutyl, butoxymethyl, butoxyethyl, butoxypropyl, butoxybutyl, hydroxycarbonyl, cyano, phenoxy, benzyloxy;
-A-A- represents the group —CHR¹—CHR²— or —CR¹═CR²—;
where R¹ and R² are independently selected from the group consisting of hydrogen, fluoro, chloro, bromo, iodo, methyl, ethyl, propyl, butyl, methoxy, ethoxy, propoxy, butoxy, acetyl, propionyl, butyryl, hydroxymethyl, hydroxyethyl, hydroxypropyl, hydroxybutyl, methoxymethyl, methoxyethyl, methoxypropyl, methoxybutyl, ethoxymethyl, ethoxyethyl, ethoxypropyl, ethoxybutyl, propoxymethyl, propoxyethyl, propoxypropyl, propoxybutyl, butoxymethyl, butoxyethyl, butoxypropyl, butoxybutyl, hydroxycarbonyl, methoxycarbonyl, ethoxycarbonyl, propoxycarbonyl, butoxycarbonyl, acetoxymethyl, acetoxyethyl, acetoxypropyl, acetoxybutyl, propionyloxymethyl, propionyloxyethyl, butyryloxymethyl, butyryloxyethyl, cyano, phenoxy and benzoxy;
or R¹ and R² together with the carbons of the steroid nucleus to which they are attached form a (saturated) cyclopropylene, cyclobutylene, cyclopentylene, cyclohexylene or cycloheptylene group;
-B-B- represents the group —CHR¹⁵—CHR¹⁶—, —CR¹⁵═CR¹⁶— or an α- or β-oriented group: 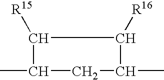
where R¹⁵ and R¹⁶ are independently selected from the group consisting of hydrogen, fluoro, chloro, bromo, iodo, methyl, ethyl, propyl, butyl, methoxy, ethoxy, propoxy, butoxy, acetyl, propionyl, butyryl, hydroxymethyl, hydroxyethyl, hydroxypropyl, hydroxybutyl, methoxymethyl, methoxyethyl, methoxypropyl, methoxybutyl, ethoxymethyl, ethoxyethyl, ethoxypropyl, ethoxybutyl, propoxymethyl, propoxyethyl, propoxypropyl, propoxybutyl, butoxymethyl, butoxyethyl, butoxypropyl, butoxybutyl, hydroxycarbonyl, methoxycarbonyl, ethoxycarbonyl, propoxycarbonyl, butoxycarbonyl, acetoxymethyl, acetoxyethyl, acetoxypropyl, acetoxybutyl, propionyloxymethyl, propionyloxyethyl, butyryloxymethyl, butyryloxyethyl, cyano, phenoxy and benzoxy;
or R¹⁵ and R¹⁶, together with the C-15 and C-16 carbons of the steroid nucleus to which R¹⁵ and R¹⁶ are respectively attached, form a cyclopropylene, cyclobutylene, cyclopentylene, cyclohexylene, cycloheptylene group;
-D-D- represents the group 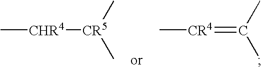
where R⁴ and R⁵ are independently selected from the group consisting of hydrogen, fluoro, chloro, bromo, iodo, methyl, ethyl, propyl, butyl, methoxy, ethoxy, propoxy, butoxy, acetyl, propionyl, butyryl, hydroxymethyl, hydroxyethyl, hydroxypropyl, hydroxybutyl, methoxymethyl, methoxyethyl, methoxypropyl, methoxybutyl, ethoxymethyl, ethoxyethyl, ethoxypropyl, ethoxybutyl, propoxymethyl, propoxyethyl, propoxypropyl, propoxybutyl, butoxymethyl, butoxyethyl, butoxypropyl, butoxybutyl, hydroxycarbonyl, methoxycarbonyl, ethoxycarbonyl, propoxycarbonyl, butoxycarbonyl, acetoxymethyl, acetoxyethyl, acetoxypropyl, acetoxybutyl, propionyloxymethyl, propionyloxyethyl, butyryloxymethyl, butyryloxyethyl, cyano, phenoxy and benzoxy; or R⁴ and R⁵ together with the carbons of the steroid backbone to which they are attached form a cyclopropylene cyclobutylene, cyclopentylene, cyclohexylene, cycloheptylene group;
-G-J- represents the group 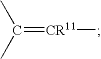
where R¹¹ is selected from the group consisting of hydrogen, methyl, ethyl, propyl, butyl, octyl, decyl, 5-fluoropentyl, 6-chlorohexyl, phenyl, p-tolyl, o-tolyl;
-E-E- represents the group —CHR⁶—CHR⁷— or —CR⁶═CR⁷—, wherein R⁶ is selected from the group consisting of hydrogen, fluoro, chloro, bromo, iodo, methyl, ethyl, propyl, butyl, methoxy, ethoxy, propoxy, butoxy, acetyl, propionyl, butyryl, hydroxymethyl, hydroxyethyl, hydroxypropyl, hydroxybutyl, methoxymethyl, methoxyethyl, methoxypropyl, methoxybutyl, ethoxymethyl, ethoxyethyl, ethoxypropyl, ethoxybutyl, propoxymethyl, propoxyethyl, propoxypropyl, propoxybutyl, butoxymethyl, butoxyethyl, butoxypropyl, butoxybutyl, hydroxycarbonyl, methoxycarbonyl, ethoxycarbonyl, propoxycarbonyl, butoxycarbonyl, acetoxymethyl, acetoxyethyl, acetoxypropyl, acetoxybutyl, propionyloxymethyl, propionyloxyethyl, butyryloxymethyl, butyryloxyethyl, cyano, phenoxy and benzoxy; and
R⁷ is selected from the group consisting of hydrogen, hydroxyl, protected hydroxyl, fluoro, chloro, bromo, iodo, methyl, ethyl, propyl, butyl, cyclopropyl, cyclobutyl, cyclopentyl, cyclohexyl, cycloheptyl, methoxy, ethoxy, propoxy, butoxy, acetyl, propionyl, butyryl, hydroxymethyl, hydroxyethyl, hydroxypropyl, hydroxybutyl, methoxymethyl, methoxyethyl, methoxypropyl, methoxybutyl, ethoxymethyl, ethoxyethyl, ethoxypropyl, ethoxybutyl, propoxymethyl, propoxyethyl, propoxypropyl, propoxybutyl, butoxymethyl, butoxyethyl, butoxypropyl, butoxybutyl, hydroxycarbonyl, methoxycarbonyl, ethoxycarbonyl, propoxycarbonyl, butoxycarbonyl, acetoxymethyl, acetoxyethyl, acetoxypropyl, acetoxybutyl, propionyloxymethyl, propionyloxyethyl, butyryloxymethyl, butyryloxyethyl, cyano, phenoxy, benzoxy, pyrrolyl, imidazolyl, thiazolyl, pyridyl, pyrimidyl, oxazolyl, acetylthio, furyl, substituted furyl, thienyl and substituted thienyl;
or R⁶ and R⁷, together with the C-6 and C-7 carbons of the steroid nucleus to which R⁶ and R⁷ are respectively attached, form a (saturated) cyclopropylene cyclobutylene, cyclopentylene, cyclohexylene, cycloheptylene group.

In many embodiments,

R¹² is selected from the group consisting of hydrogen, fluoro, chloro, bromo, iodo, fluoromethyl, fluoroethyl, fluoropropyl, fluorobutyl, chloromethyl, chloroethyl, chloropropyl, chlorobutyl, bromomethyl, bromoethyl, bromopropyl, bromobutyl, iodomethyl, iodoethyl, iodopropyl, iodobutyl, hydroxy, methyl, ethyl, straight, branched or cyclic propyl and butyl; methoxy, ethoxy, propoxy, butoxy, hydroxymethyl, hydroxyethyl, hydroxypropyl, hydroxybutyl, and cyano;
R¹⁰ and R¹³ are methyl, typically β-methyl;
-A-A- represents the group —CH₂—CH₂— or —CH═CH—;
-B-B- represents the group —CHR¹⁵—CHR¹⁶—, —CR¹⁵═CR¹⁶— or an α- or β-oriented group: 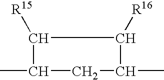
where R¹⁵ and R¹⁶ are independently selected from the group consisting of hydrogen, fluoro, chloro, bromo, iodo, methyl, ethyl, propyl, butyl, methoxy, ethoxy, propoxy, butoxy, acetyl, propionyl, butyryl, hydroxymethyl, hydroxyethyl, hydroxypropyl, hydroxybutyl and cyano;
or R¹⁵ and R¹⁶, together with the C-15 and C-16 carbons of the steroid nucleus to which R¹⁵ and R¹⁶ are respectively attached, form a cyclopropylene, cyclobutylene, cyclopentylene, cyclohexylene, cycloheptylene group;
-D-D- represents the group 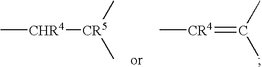
where R⁴ and R⁵ are independently selected from the group consisting of hydrogen, fluoro, chloro, bromo, iodo, methyl, ethyl, propyl, butyl, methoxy, ethoxy, propoxy, butoxy, acetyl, propionyl, butyryl, hydroxymethyl, hydroxyethyl, hydroxypropyl, hydroxybutyl and cyano;
-E-E- represents the group —CHR⁶—CHR⁷— or —CR⁶═CR⁷—, wherein R⁶ is selected from the group consisting of hydrogen, fluoro, chloro, bromo, iodo, methyl, ethyl, propyl, butyl, methoxy, ethoxy, propoxy, butoxy, acetyl, propionyl, butyryl, hydroxymethyl, hydroxyethyl, hydroxypropyl, hydroxybutyl and cyano; and
R⁷ is selected from the group consisting of hydrogen, hydroxyl, protected hydroxyl, fluoro, chloro, bromo, iodo, methyl, ethyl, propyl, butyl, cyclopropyl, cyclobutyl, cyclopentyl, cyclohexyl, cycloheptyl, methoxy, ethoxy, propoxy, butoxy, acetyl, propionyl, butyryl, hydroxymethyl, hydroxyethyl, hydroxypropyl, hydroxybutyl, cyano, furyl, thienyl, substituted furyl and substituted thienyl;
or R⁶ and R⁷, together with the C-6 and C-7 carbons of the steroid nucleus to which R⁶ and R⁷ are respectively attached, form a (saturated) cyclopropylene cyclobutylene, cyclopentylene, cyclohexylene, cycloheptylene group.

In various preferred embodiments, R¹² is selected from the group consisting of hydrogen, halo, hydroxy, alkyl, alkoxy, hydroxyalkyl, alkoxyalkyl, hydroxycarbonyl, cyano and aryloxy;
R¹⁰ and R¹³ are methyl, particularly β-methyl;
-A-A- represents the group —CH₂—CH₂—;
-B-B- represents the group —CHR¹⁵—CHR¹⁶—; where R¹⁵ and R¹⁶ are hydrogen;
or R¹⁵ and R¹⁶, together with the C-15 and C-16 carbons of the steroid nucleus to which they are respectively attached, form a (saturated) cycloalkylene group;
-D-D- represents the group: 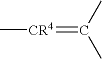
where R⁴ is hydrogen;
-E-E- represents the group —CHR⁶—CHR⁷—; where R⁶ is hydrogen;
where R⁷ is selected from the group consisting of hydrogen, furyl, substituted furyl, thienyl, substituted thienyl and acetylthio;
or R⁶ and R⁷, together with the C-6 and C-7 carbons of the steroid nucleus to which they are respectively attached, form a (saturated) cycloalkylene group;
-G-J- represents the group 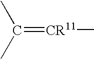
where R¹¹ is hydrogen.

Unless stated otherwise, organic radicals referred to as “lower” in the present disclosure contain at most 7, and preferably from 1 to 4, carbon atoms.

A lower alkoxycarbonyl radical is preferably one derived from an alkyl radical having from 1 to 4 carbon atoms, such as methyl, ethyl, propyl, isopropyl, butyl, isobutyl, sec-butyl and tert-butyl; especially preferred are methoxycarbonyl, ethoxycarbonyl and isopropoxycarbonyl. A lower alkoxy radical is preferably one derived from one of the above-mentioned C₁-C₄ alkyl radicals, especially from a primary C₁-C₄ alkyl radical; especially preferred is methoxy. A lower alkanoyl radical is preferably one derived from a straight-chain alkyl having from 1 to 7 carbon atoms; especially preferred are formyl and acetyl.

A methylene bridge in the 15,16-position is preferably β-oriented.

A preferred class of compounds that may be produced in accordance with the methods of the invention are the 20-spiroxane compounds described in U.S. Pat. No. 4,559,332, i.e., those corresponding to Formula IA:

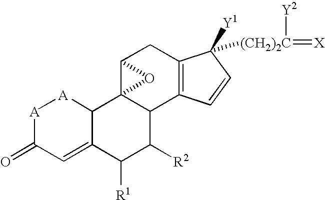

Preferably, 20-spiroxane compounds produced by the novel methods of the invention are those of Formula I in which Y¹ and Y² together represent the oxygen bridge —O—.

Especially preferred compounds of the formula I are those in which X represents oxo. Of compounds of the 20-spiroxane compounds of Formula IA in which X represents oxo, there are most especially preferred those in which Y¹ together with Y² represents the oxygen bridge —O—.

Especially preferred compounds of the formula I and IA are, for example, the following:
9α,11α-epoxy-7α-methoxycarbonyl-20-spirox-4-ene-3,21-dione,
9α,11α-epoxy-7α-ethoxycarbonyl-20-spirox-4-ene-3,21-dione,
9α,11α-epoxy-7α-isopropoxycarbonyl-20-spirox-4-ene-3,21-dione,
and the 1,2-dehydro analog of each of the compounds;
9α,11α-epoxy-6α,7α-methylene-20-spirox-4-ene-3,21-dione,
9α,11α-epoxy-6β,7β-methylene-20-spirox-4-ene-3,21-dione,

9α,11α-epoxy-6β,7β; 15β,16β-bismethylene-20-spirox-4-ene-3,21-dione,
and the 1,2-dehydro analog of each of these compounds;
9α,11α-epoxy-7α-methoxycarbonyl-17β-hydroxy-3-oxo-pregn-4-ene-21-carboxylic acid,
9α,11α-epoxy-7α-ethoxycarbonyl-17β-hydroxy-3-oxo-pregn-4-ene-21-carboxylic acid,
9α,11α-epoxy-7α-isopropoxycarbonyl-17β-hydroxy-3-oxo-pregn-4-ene-21-carboxylic acid,
9α,11α-epoxy-17β-hydroxy-6α,7α-methylene-3-oxo-pregn-4-ene-21-carboxylic acid,
9α,11α-epoxy-17β-hydroxy-6β,7β-methylene-3-oxo-pregn-4-ene-21-carboxylic acid,
9α,11α-epoxy-17β-hydroxy-6β,7β; 15β, 16β-bismethylene-3-oxo-pregn-4-ene-21-carboxylic acid,
and alkali metal salts, especially the potassium salt or ammonium salt of each of these acids, and also a corresponding 1,2-dehydro analog of each of the mentioned carboxylic acids or of a salt thereof;
9α,11α-epoxy-15β,16β-methylene-3,21-dioxo-20-spirox-4-ene-7α-carboxylic acid methyl ester, ethyl ester and isopropyl ester,
9α,11α-epoxy-15β,16β-methylene-3,21-dioxo-20-spiroxa-1,4-diene-7α-carboxylic acid methyl ester, ethyl ester and isopropyl ester,
9α,11α-epoxy-3-oxo-20-spirox-4-ene-7α-carboxylic acid methyl ester, ethyl ester and isopropyl ester,
9α,11α-epoxy-6β,6β-methylene-20-spirox-4-en-3-one,
9α,11α-epoxy-6β,7β; 15β,16β-bismethylene-20-spirox-4-en-3-one,
9α,11α-epoxy,17β-hydroxy-17α(3-hydroxy-propyl)-3-oxo-androst-4-ene-7α-carboxylic acid methyl ester, ethyl ester and isopropyl ester,
9α,11α-epoxy,17β-hydroxy-17α-(3-hydroxypropyl)-6α, 7α-methylene-androst-4-en-3-one,
9α,11α-epoxy-17β-hydroxy-17α-(3-hydroxypropyl)-6β,7β-methylene-androst-4-en-3-one,
9α,11α-epoxy-17α-hydroxy-17α-(3-hydroxypropyl)-6β,7β; 15β,16β-bismethylene-androst-4-en-3-one,
   including 17α-(3-acetoxypropyl) and 17α-(3-formyloxypropyl) analogs of the mentioned androstane compounds,
   and also 1,2-dehydro analogs of all the mentioned compounds of the androst-4-en-3-one and 20-spirox-4-en-3-one series.

The chemical names of the compounds of Formulas I and IA, and of analog compounds having the same characteristic structural features, are derived according to current nomenclature in the following manner: for compounds in which Y¹ together with Y² represents —O—, from 20-spiroxane (for example a compound of the Formula IA in which X represents oxo and Y¹ together with Y² represents —O— is derived from 20-spiroxan-21-one); for those in which each of Y¹ and Y² represents hydroxy and X represents oxo, from 17β-hydroxy-17α-pregnene-21-carboxylic acid; and for those in which each of Y¹ and Y² represents hydroxy and X represents two hydrogen atoms, from 17β-hydroxy-17α-(3-hydroxypropyl)-androstane. Since the cyclic and open-chain forms, that is to say lactones and 17β-hydroxy-21-carboxylic acids and their salts, respectively, are so closely related to each other that the latter may be considered merely as a hydrated form of the former, there is to be understood hereinbefore and hereinafter, unless specifically stated otherwise, both in end products of the formula I and in starting materials and intermediates of analogous structure, in each case all the mentioned forms together.

Exemplary substrates for this reaction include Δ-9,11-canrenone, and

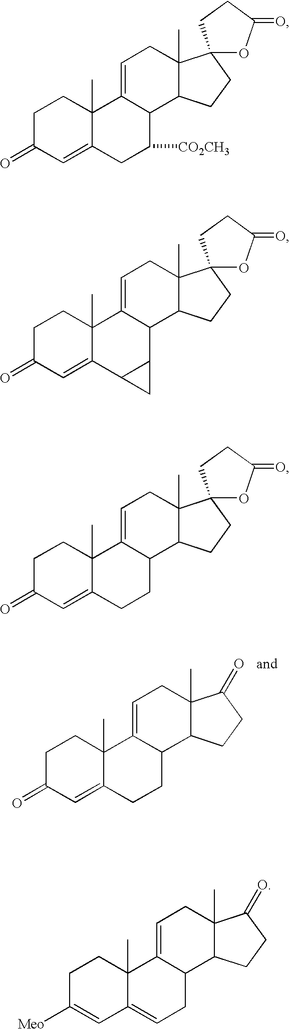

Generally, the epoxidation process of the invention is conducted in accordance with the procedure described in U.S. Pat. No. 4,559,332, as more particularly described in U.S. Pat. No. 5,981,744, col. 40, line 38 to col. 45, line 15 and in Examples 26-28 and 42-51. See also U.S. Pat. No. 6,610,844. The 4,559,332, 5,981,744 and 6,610,844 patent documents are expressly incorporated herein by reference.

In the epoxidation process as described in these references, a solution of Δ^{9,11} substrate in a suitable solvent is contacted with an aqueous hydrogen peroxide composition in the presence of an activator such as, for example, trichloracetonitrile or, preferably, trichloroacetamide. With the goal of assuring complete conversion of the substrate to the 9,11-epoxide, the epoxidation reaction as described in the above-cited references is typically conducted at a molar charge ratio of ≧10 moles hydrogen peroxide per mole steroid substrate.

It has now been discovered that the epoxidation reaction can be conducted at a significantly lower ratio of hydrogen peroxide to Δ^{9,11} substrate than is taught or exemplified in U.S. Pat. Nos. 4,559,332, 5,981,744 or U.S. Pat. No. 6,610,844. Operation at a relatively low peroxide to substrate ratio provides the option of achieving any of several potential advantages, as discussed hereinbelow.

In carrying out the reaction, preferably the solution of substrate, together with the activator and a buffer are first charged to a reaction vessel comprising an epoxidation reaction zone, and an aqueous solution of hydrogen peroxide added thereto. Preferably, a solvent for the steroid substrate is selected in which the solubility of the steroid substrate and epoxidized steroid product is reasonably high, preferably at least about 10 wt. %, more preferably at least about 20 wt. %, but in which the solubility of water is low, preferably less than about 1 wt. %, more preferably less than about 0.5 wt. %. In such embodiments, an epoxidation reaction zone comprising a two phase liquid reaction medium that is established within the reaction vessel, with the substrate in the organic phase and hydrogen peroxide in the aqueous phase. Epoxidation of the substrate in the two phase medium produces a reaction mass containing the epoxidized steroid reaction product substantially within the solvent phase. Without being held to a particular theory, it is believed that the reaction occurs in the organic phase or at the interface between the phases, and that more than a very minor water content in the organic phase effectively retards the reaction.

After the solution of steroid is introduced into the reactor, the entire peroxide solution may be added over a short period of time before reaction is commenced, e.g., within 2 to 30 minutes, more typically 5 to 20 minutes. Where the strength of the peroxide solution as supplied to the reactor is greater than the concentration to be established at the outset of the reaction, water may be charged and mixed with the organic phase prior to addition of peroxide, water being added in a volume which thereafter dilutes the peroxide concentration to the level desired at the outset of the reaction. In those embodiments wherein hydrogen peroxide is introduced at the beginning of the reaction cycle, the solvent phase and added aqueous peroxide solution are preferably maintained at a relatively low temperature, more preferably, lower than about 25° C., typically lower than about 20° C., more typically in the range of about −5° to about 15° C., as the peroxide is introduced.

Reaction then proceeds under agitation. Preferably the reaction is conducted under an inert atmosphere, preferably by means of a nitrogen purge of the reactor head space.

Generically, the peroxide activator may correspond to the formula:
R^{o}C(O)NH₂
where R^{o} is a group having an electron withdrawing strength (as measured by sigma constant) at least as high as that of the monochloromethyl group. Preferably, the promoter comprises trichloroacetonitrile, trichloracetamide, or a related compound corresponding to the formula: 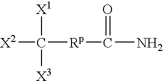
where X¹, X², and X³ are independently selected from among halo, hydrogen, alkyl, haloalkyl and cyano and cyanoalkyl, and RP is selected from among arylene and —(CX⁴X⁵)ₙ—, where n is 0 or 1, at least one of X¹, X², X³, X⁴ and X⁵ being halo or perhaloalkyl. Where any of X¹, X², X³, X⁴ or X⁵ is not halo, it is preferably haloalkyl, most preferably perhaloalkyl. Particularly preferred activators include those in which n is O and at least two of X¹, X² and X³ are halo; or in which all of X¹, X², X³, X⁴ and X⁵ are halo or perhaloalkyl. Each of X¹, X², X³, X⁴ and X⁵ is preferably Cl or F, most preferably Cl, though mixed halides may also be suitable, as may perchloralkyl or perbromoalkyl and combinations thereof.

Other suitable promoters include hexafluoroacetone dicyclohexylcarbodiimide.

The buffer stabilizes the pH of the reaction mass. Without being bound to a particular theory, the buffer is further believed to function as a proton transfer agent for combining the peroxide anion and promoter in a form which reacts with the Δ^{9,11} substrate to form the 9,11-epoxide. It is generally desirable that the reaction be conducted at a pH in the range of about 5 to about 8, preferably about 6 to about 7. Suitable compounds which may function both as a buffer and as a proton transfer agent include dialkali metal phosphates, and alkali metal salts of dibasic organic acids, such as Na citrate or K tartrate.

Especially favorable results are obtained with a buffer comprising dipotassium hydrogen phosphate, and/or with a buffer comprising a combination of dipotassium hydrogenphosphate and potassium dihydrogen phosphate in relative proportions of between about 1:4 and about 2:1, most preferably in the range of about 2:3. Borate buffers can also be used, but generally give slower conversions than dipotassium phosphate or KH₂PO₄ or K₂HPO₄/KH₂PO₄ mixtures. Whatever the makeup of the buffer, it should provide a pH in the range indicated above. Aside from the overall composition of the buffer or the precise pH it may impart, it has been observed that the reaction proceeds much more effectively if at least a portion of the buffer is comprised of dibasic hydrogenphosphate ion. It is believed that this ion may participate essentially as a homogeneous catalyst in the formation of an adduct or complex comprising the promoter and hydroperoxide ion, the generation of which may in turn be essential to the overall epoxidation reaction mechanism. Thus, the quantitative requirement for dibasic hydrogenphosphate (preferably from K₂HPO₄) may be only a small catalytic concentration. Generally, it is preferred that a dibasic hydrogenphosphate be present in a proportion of at least about 0.1 equivalents, e.g., between about 0.1 and about 0.3 equivalents, per equivalent substrate.

After addition of the peroxide solution is substantially complete, the temperature may be raised, e.g., into the range of 15° to 50° C., more typically 20° to 40° C. to enhance the rate of the reaction and the conversion of substrate to epoxide. Optionally, the peroxide solution can be added progressively over the course of the reaction, in which case the temperature of the reaction mass is preferably maintained in a range of about 15° to about 50° C., more preferably between about 20° and about 40° C. as the reaction progresses. In either case, the reaction rate in the two phase reaction medium is ordinarily mass transfer limited, requiring modest to vigorous agitation to maintain a satisfactory reaction rate. In a batch reactor, completion of the reaction may require from 3 to 24 hours, depending on the temperature and intensity of agitation.

The decomposition of hydrogen peroxide is an exothermic reaction. At ordinary reaction temperatures the rate of decomposition is small to negligible, and the heat generated is readily removed by cooling the reaction mass under temperature control. However, if the reaction cooling system or temperature control system fails, e.g., by loss of agitation, the rate of decomposition can be accelerated by the resulting increase in temperature of the reaction mass, which can in turn accelerate the rate of autogenous reaction heating. Where the initial molar ratio of peroxide to steroid substrate is in the range described in U.S. Pat. No. 4,559,332, U.S. Pat. No. 5,981,744 or U.S. Pat. No. 6,610,844, i.e., in the range of 10:1 or higher, autogenous heating as resulting from loss of cooling can reach a temperature at which the decomposition becomes autocatalytic, and thus very rapid and uncontrolled, resulting in potential eruption of the reaction mass. If the temperature is high enough, destructive oxidation of the steroid substrate may generate additional reaction heat, further accelerating the rate of temperature increase and the severity of the resulting eruption. Events other than loss of agitation can also potentially destabilize the peroxide and result in an exotherm that leads to uncontrolled decomposition. For example, contaminants such as rust or other source of transition metals in the peroxide or substrate solutions may catalyze a rapid or uncontrolled release of oxygen from the aqueous phase.

It has now been discovered that the epoxidation reaction can be conducted at a significantly lower ratio of peroxide to Δ^{9,11} substrate than is taught or exemplified in U.S. Pat. Nos. 4,559,332, 5,981,744 or U.S. Pat. No. 6,610,844, thereby reducing the risk of uncontrolled decomposition of the peroxide. More particularly, it has been discovered that the reaction can be conducted at a charge ratio between about 2 and about 7 moles, preferably between about 2 and about 6 moles, more preferably between about 3 and about 5 moles hydrogen peroxide per mole Δ^{9,11} substrate. Operation at such relatively low ratios of peroxide to substrate reduces the extent to which the reaction mass may be heated by autogenous decomposition of the peroxide. Preferably, the peroxide to substrate ratio is low enough so that the maximum temperature attainable by autogenous heating is lower than the threshold temperature for autocatalytic decomposition, which may entirely preclude decomposition of the peroxide from reaching the stage at which an eruption of the reaction mass could result. Operation at the above described charge ratios makes this feasible.

Further protection against uncontrolled reaction is provided where the epoxidation reaction is conducted at a relatively modest temperature below the temperature of incipient decomposition of the peroxide, or where the rate of decomposition is relatively slow. Thus, in the event of a process upset which results in accumulation of unreacted hydrogen peroxide, little autogenous heating can occur, at least initially, so that, even after loss of agitation, reactor cooling capacity remains sufficient under natural circulation to maintain the temperature of the reaction mass in a safe range, or at least process operators are afforded ample time to take corrective measures before conditions for an uncontrolled autocatalytic decomposition are approached. For this purpose, it is preferred that the epoxidation reaction be carried out at a temperature in the range of about 0° to 50° C., more preferably in the range of about 20° to about 40° C.

Still further protection against uncontrolled reaction is afforded by conducting the epoxidation reaction in a liquid reaction medium comprising a solvent having a boiling point at the reaction pressure that is well below the autocatalytic decomposition temperature of the peroxide, and preferably only modestly higher than the reaction temperature. Preferably, the boiling point of the organic phase of the reaction mixture is no greater than about 60° C., preferably not greater than about 50° C. Preferably, the selected solvent does not boil from the reaction mass at the reaction temperature, but is rapidly vaporized if the temperature increases by a modest increment from about 10 centigrade degrees to about 50 centigrade degrees, whereby the heat of vaporization serves as a heat sink precluding substantial heating of the reaction mass until the solvent shall have been substantially driven out of the reaction zone. Where the reaction is conducted under atmospheric pressure at a temperature in the aforesaid ranges, a variety of solvents are available which meet these criteria, and are also suitable for the epoxidation reaction. These include methylene chloride (atmos. b.p.=39.75° C.), dichloroethane (atmospheric b.p.=83° C., and methyl t-butyl ether (b.p.=55° C.).

The water content of the reaction mass also serves as a substantial sensible heat sink. Where the reaction is conducted at, near or below atmospheric pressure, the water content of the aqueous hydrogen peroxide solution serves as a potentially much larger heat sink, though it is generally preferred to avoid conditions under which substantial steam generation occurs since this may also result in eruption of the reaction mass, albeit much less violent than that which results from autocatalytic decomposition of a peroxide compound.

Thus, in one aspect, the present invention comprises conducting the epoxidation reaction in a liquid reaction medium, preferably comprising a solvent for the steroid, which contains the steroid substrate and peroxide in such absolute and relative proportions, and at a relatively modest initial epoxidation reaction temperature, such that the decomposition of the peroxide content of the reaction mass in stoichiometric excess vs. the substrate charge does not, and preferably cannot, produce an exotherm effective to initiate autocatalytic decomposition of peroxide compound, or at least not to cause an uncontrolled autocatalytic decomposition thereof. To protect against an uncontrolled decomposition at any time during the epoxidation cycle, it is further preferred that the aforesaid combination of conditions be such that decomposition of the entire peroxide content of the reaction mass, at any time during the course of the reaction, cannot produce an exotherm effective to initiate autocatalytic decomposition of peroxide compound, or at least not to cause an uncontrolled autocatalytic decomposition thereof. Optimally, the combination of substrate concentration, peroxide compound concentration and initial temperature are such that decomposition of the stoichiometeric excess, or of the entire peroxide compound charge, cannot produce an exotherm sufficient to initiate autocatalytic decomposition, or at least not to cause an uncontrolled autocatalytic decomposition, even under adiabatic conditions, i.e., upon loss of cooling in a well-insulated reactor.

The peroxide content of the aqueous phase, as established at the outset of the epoxidation reaction, is preferably between about 25% and about 50% by weight, more preferably between about 25% and about 35% by weight, and the initial concentration of Δ^{9,11} steroid substrate in the organic phase is between about 3% and about 25% by weight, more preferably between about 7% and about 15% by weight. Preferably, components effective to promote the epoxidation reaction such as, for example, trichloroacetonitrile or trichloroacetamide, together with a phosphate salt such as a dialkali hydrogen phosphate, are charged to the reactor with the steroid solution, prior to addition of the aqueous peroxide. The molar ratio of peroxide to phosphate is preferably maintained in the range between about 10:1 and about 100:1, more preferably between about 20:1 and about 40:1. The initial trichloroacetamide or trichloroacetonitrile concentration is preferably maintained at between about 2 and about 5 wt. %, more preferably between about 3 and about 4 wt. %, in the organic phase; or in a molar ratio to the steroid substrate between about 1.1 and about 2.5, more preferably between about 1.2 and about 1.6. The volumetric ratio of the aqueous phase to the organic phase ultimately introduced into the reactor is preferably between about 10:1 and about 0.5:1, more preferably between about 7:1 and about 4:1. As mentioned above, and again without being held to a particular theory, it is believed that the epoxidation reaction occurs in the organic phase or at the interface between the phases. In any event, the reaction mass is preferably agitated vigorously to promote transfer of peroxide to the organic phase, or at least to the interface. A high rate of mass transfer is desired both to promote the progress of the reaction, thereby shortening batch reaction cycles and enhancing productivity, and to minimize the inventory of peroxide in the reaction vessel at any given rate of addition of aqueous peroxide solution to the reaction mass. Thus, in various preferred embodiments of the invention, the agitation intensity is at least about 10 hp/1000 gal. (about 2 watts/liter, typically from about 15 to about 25 hp/1000 gal. (about 3 to about 5 watts/liter). The epoxidation reactor is also provided with cooling coils, a cooling jacket, or an external heat exchanger through which the reaction mass is circulated for removal of the heat of the epoxidation reaction, plus any further increment of heat resulting from decomposition of the peroxide.

After completion of the epoxidation reaction, unreacted hydrogen peroxide in the aqueous phase is preferably decomposed under controlled conditions under which release of molecular oxygen is minimized or entirely avoided. A reducing agent such as an alkali metal sulfite or alkali metal thiosulfate is effective for promoting the decomposition. Preferably, the aqueous phase of the final reaction mass, which comprises unreacted peroxide, is separated from the organic phase, which comprises a solution of 9,11-epoxidized steroid product in the reaction solvent. The aqueous phase may then be “quenched” by contact of the peroxide contained therein with the reducing agent.

Where the molar charge ratio of peroxide to steroid substrate is in the range of, for example, 3 to 5, and the initial concentration of a peroxide in the aqueous phase is in the range of about 7 to about 9 molar concentration (i.e., 25% to 30% by weight in the case of hydrogen peroxide), the spent aqueous peroxide solution at the end of the reaction contains about 4-6 molar concentration % peroxide (between about 15 and about 21% by weight for hydrogen peroxide). Prior to phase separation, the aqueous phase may be diluted with water to reduce the peroxide concentration and thereby the likelihood and extent of any exotherm resulting from decomposition during the phase separation and/or transfer of the aqueous phase, such as transfer to another vessel for quenching with a reducing agent. For example, sufficient water may be added to reduce the concentration of hydrogen peroxide in the spent aqueous phase to between about 2% and about 10% by weight, more preferably between about 2% and about 5% by weight.

Quenching may be effected by adding the spent aqueous peroxide solution, or a dilution thereof, to a vessel containing an aqueous solution of the reducing agent, or vice-versa. According to one alternative, the organic phase may be transferred to a separate vessel upon separation from the aqueous phase, and the aqueous phase allowed to remain in the reaction vessel. The solution of the reducing agent may then be added to the diluted or undiluted aqueous phase in the reaction vessel to effect reduction of the residual peroxide. Alternatively, the diluted or undiluted peroxide solution may be added over time to a vessel to which an appropriate volume of reducing agent solution has initially been charged. Where the reducing agent is an alkali metal sulfite, the sulfite ion reacts with the peroxide to form sulfate ion and water.

The decomposition reaction is highly exothermic. Decomposition is preferably conducted at a temperature controlled in the range of between about 20° C. and about 50° C. by transfer of heat from the aqueous mass in which the decomposition proceeds. For this purpose, the quenching reactor may be provided with cooling coils, a cooling jacket, or an external heat exchanger through which the quench reaction mass may be circulated, for transfer of decomposition reaction heat to a cooling fluid. The quenching mass is preferably subjected to moderate agitation to maintain uniform distribution of reducing agent, uniform temperature distribution, and rapid heat transfer.

Where the reducing agent is added to the spent peroxide solution, addition is preferably carried out at a rate controlled to maintain the temperature of the quench reaction mass in the aforesaid range, thereby to effect controlled decomposition of the peroxide.

The alternative process, i.e., the process wherein the peroxide solution is added to the reducing agent solution, avoids the presence of a large inventory of peroxide that might otherwise be subject to autocatalytic decomposition as triggered by the addition of a decomposition agent thereto. However, this alternative requires transfer of the spent peroxide solution while the reverse alternative allows the peroxide solution to be retained in the epoxidation reactor while only the organic phase of the reaction mass and the reducing agent solution need to be transferred. Regardless of which alternative is followed, the quench reaction is preferably conducted in the temperature range specified above.

For purposes of the quenching reaction, the aqueous quench solution charged to the quenching reaction zone preferably contains between about 12 wt % and about 24 wt. %, more preferably between about 15 wt % and about 20 wt. %, of a reducing agent such as Na sulfite, Na bisulfite, etc. The volume of quench solution is preferably sufficient so that the reducing agent contained therein is in stoichiometric excess with respect to the peroxide content of the aqueous phase to be quenched. The volumetric ratio of quench solution that is mixed with the peroxide solution may typically vary from about 1.2 to about 2.8, more typically from about 1.4 to about 1.9 after preliminary water dilution of the spent aqueous peroxide solution.

Typically, residual organic solvent may have remained in the reactor after the initial phase separation, and have become entrained in the aqueous phase during the quenching reaction. Also, the quenched aqueous phase may contain a salt of trichloroacetic acid, formed as a by-product of the epoxidation reaction when trichloroacetamide is used as a promoter. Before disposal of the quenched aqueous phase, entrained reaction solvent is preferably removed therefrom, e.g., by solvent stripping. If a solvent such as methylene chloride is entrained in the quench reaction mixture, and the aqueous phase thereof contains trichloroacetate, the aqueous phase is preferably heated prior to solvent stripping in order to decarboxylate the trichloroacetate. Decarboxylation of the trichloroacetate may be achieved by heating to a temperature of, e.g., 70° C. or higher. If trichloroacetate is not removed, it can decompose during solvent stripping to produce chloroform and carbon dioxide.

After separation from the aqueous phase of the reaction mass, the organic phase is preferably washed with water to remove unreacted peroxide and any inorganic contaminants. For elimination of residual peroxide it may be useful for the wash water to contain a reducing agent. For example, the organic phase may be contacted with an aqueous wash solution having a pH in the range of 4 to 10 and containing typically 0.1 to 5 mole % reducing agent, preferably about 0.2 to about 0.6 mole % reducing agent (such as, e.g., 6 to 18% aqueous solution of Na sulfite), in a convenient volumetric ratio of wash solution to organic phase between about 0.05:1 to about 0.3:1. After separation of the spent reducing agent wash from the organic phase, the organic phase is preferably washed sequentially with a dilute caustic solution (e.g., 0.2% to 6% by weight NaOH in a volumetric ratio to the organic phase between about 0.1 to about 0.3) followed by either a water wash or a dilute acid solution (for example, a 0.5 to 2 wt. % HCl solution in a volumetric ratio to the organic phase between about 0.1 and about 0.4). A final wash with further Na bisulfite or Na metabisulfite or Na sulfite solution may also be conducted.

Where the R¹¹ substituent of the product epoxide is other than hydrogen, it is generally desirable to avoid a highly acidic wash, such as an HCl wash which can expose the product to an aqueous phase having a pH of 1 or less. Where there is an alkyl substituent at the C-11 carbon, the epoxy group may destabilize under highly acidic conditions.

If a solvent such as methylene chloride is entrained in the dilute caustic wash, the aqueous phase thereof contains trichlorosodiumacetate produced from basic hydrolysis of residual trichloroacetamide, the aqueous phase is preferably heated prior to solvent stripping in order to decarboxylate the trichlorosodiumacetate. Decarboxylation of the trichlorosodiumacetate may be achieved by heating to a temperature of, e.g., 70° C. or higher. The caustic wash may be combined with the quenched aqueous phase of the reaction mixture for purposes of decarboxylation and residual solvent stripping.

The washed organic phase is concentrated by evaporation of solvent, for example, by atmospheric distillation, resulting in precipitation of steroid to form a relatively thick slurry with about 40% to about 75% by weight contained steroid. Where mother liquor from a recrystallization step is recycled, as described below, the mother liquor may be mixed with the steroid slurry, and the solvent component of the mother liquor removed by vacuum to again produce a thick slurry having a solids concentration typically in the same range as the slurry obtained by removing the reaction solvent. A solvent in which the solubility of the steroid product is relatively low, e.g., a polar solvent such as ethanol, is added to the slurry obtained from removal of reaction solvent, or to the second slurry as obtained by removal of the recrystallization mother liquor solvent. Alternative solvents include toluene, acetone, acetonitrile and acetonitrile/water. In this step, the impurities are digested into the solvent phase, thus refining the solid phase steroid product to increase its assay. Where the digestion solvent is an alcohol such as ethanol, it may be added in a volumetric ratio of ethanol to contained steroid between 6 and about 20. A portion of the ethanol and residual organic solvent are removed from the resulting mixture by distillation, yielding a slurry typically containing between about 10 wt. % and about 20 wt. % steroid product, wherein impurities and by-products are substantially retained in the solvent phase. Where the solvent is ethanol, the distillation is preferably conducted at atmospheric pressure or slightly above.

After distillation of the digestion solvent, the steroid product solids are separated from the residual slurry, e.g., by filtration. The solid product is preferably washed with the digestion solvent, and may be dried to yield a solid product substantially comprising the 9,11-epoxy steroid. Drying may advantageously be conducted with pressure or vacuum using an inert carrier gas at a temperature in the range of about 35 to about 90° C.

Either the dried solids, wet filtered solids or the residual slurry obtained after evaporation of the digestion solvent may be taken up in a solvent in which the epoxy steroid product is moderately soluble, e.g., 2-butanone (methyl ethyl ketone), methanol, isopropanol-water or acetone-water. The resulting solution may typically contain between about 3% and about 20% by weight, more typically between about 5% and about 10% by weight, steroid. The resulting solution may be filtered, if desired, and then evaporated to remove the polar solvent and recrystallize the 9,11-epoxy steroid. Where the solvent is 2-butanone, evaporation is conveniently conducted at atmospheric pressure, but other pressure conditions may be used. The resulting slurry is cooled slowly to crystallize additional steroid. For example, the slurry may be cooled from the distillation temperature (about 80° C. in the case of 2-butanone at atmospheric pressure) to a temperature at which yield of steroid product is deemed satisfactory. Production of a highly pure 9,11-epoxy steroid product of a suitable crystal size may be produced by cooling in stages and holding the temperature for a period between cooling stages. An exemplary cooling schedule comprises cooling in a first stage to a temperature in the range of 60° to 70° C., cooling in a second stage to a temperature in the range of about 45° to about 55° C., cooling in a third stage to a temperature between about 30° and about 40° C., and cooling in a final stage to a temperature between about 10° and about 20° C., with substantially constant temperature hold periods of 30 to 120 minutes between cooling stages.

The recrystallized product may then be recovered by filtration and dried. Drying may be conducted effectively at near ambient temperature. The dried product may remain solvated with the polar solvent used early in the product recovery protocol, typically ethanol. Drying and desolvation may be completed at elevated temperature under pressure or vacuum, e.g., at 75° to 95° C.

Mother liquor from the recrystallization step may be recycled for use in refining the steroid product slurry obtained from evaporative removal of the epoxidation reaction solvent, as described hereinabove.

At a charge ratio of 7 moles peroxide per mole substrate in the oxidation of the Δ^{9,11} precursor to eplerenone, decomposition of the peroxide releases only about 280 liters molecular oxygen per kg eplerenone. At a charge ratio of 4 moles peroxide per mole substrate, the oxygen release is only about 160 liters/kg eplerenone. This contrasts with a release of 400 liters/kg eplerenone at a charge ratio of 10 moles peroxide per mole substrate. By way of further example, at a charge ratio of 4 moles peroxide per mole substrate, a substrate concentration of 12% in a methylene chloride solvent, a peroxide concentration in the aqueous phase of 30%, an initial reaction temperature of 30° C., substantially at atmospheric pressure under an inert gas purge, and a reactor head space volume fraction of 15%, the maximum internal pressure that can be generated in the epoxidation reactor upon exothermic decomposition of the entire peroxide charge is about 682 psig. Moreover, even in this instance, the initial exotherm is modest enough that a reasonably skilled operator should have ample time to safely deal with loss of agitation or other process upset that could otherwise potentially lead to uncontrolled reaction.

At the relatively low peroxide to substrate ratios described herein, either significantly lesser potential evolution of oxygen can be assured at the same reactor payload that can be achieved at peroxide/substrate ratios of 10 or more; or higher reactor payloads may be achieved at the same volume of oxygen release. At constant working volume in an epoxidation reactor, both an increase in payload and a reduction in oxygen release can be achieved.

It should be understood that the epoxidation method as described above has application beyond the various schemes for the preparation of epoxymexrenone, and in fact may be used for the formation of epoxides across 9,11-olefinic double bonds in a wide variety of substrates subject to reaction in the liquid phase. Exemplary substrates for this reaction include Δ-9,11-canrenone, and 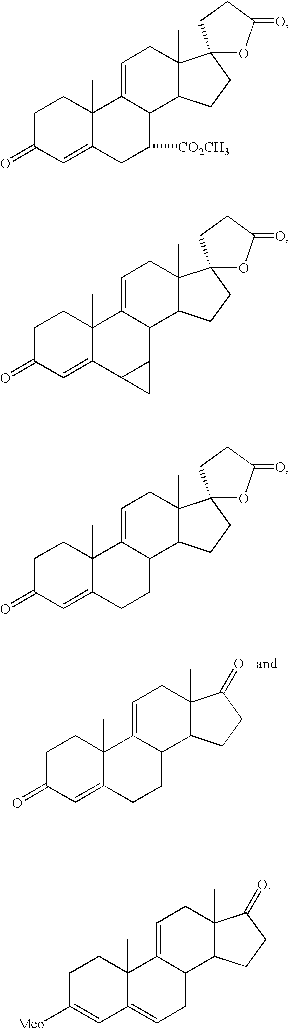
Because the reaction proceeds more rapidly and completely with trisubstituted and tetrasubstituted double bonds, it is especially effective for selective epoxidation across such double bonds in compounds that may include other double bonds where the olefinic carbons are monosubstituted, or even disubstituted.

Because it preferentially epoxidizes the more highly substituted double bonds, e.g., the 9,11-olefin, with high selectivity, the process of this invention is especially effective for achieving high yields and productivity in the epoxidation steps of the various reaction schemes described elsewhere herein.

The improved process has been shown to be particularly advantageous application to the preparation of: 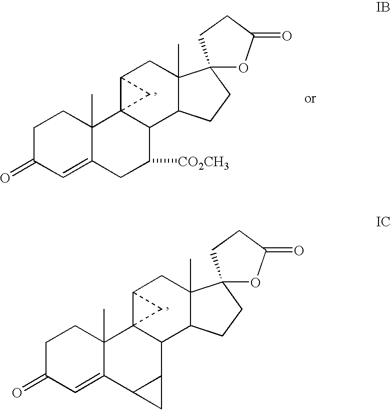
by epoxidation of:

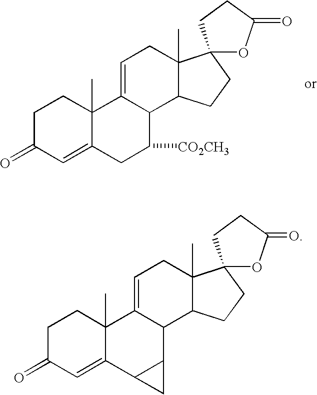

### EXAMPLE 1

### Synthesis of Methyl Hydrogen 9,11α-Epoxy-17α-hydroxy-3-oxopregn-4-ene-7α,21-dicarboxylate, γ-Lactone

Crude Δ^{9,11}-eplerenone precursor (1628 g, assaying 78.7% enester) was added to a reaction vessel with methylene chloride (6890 mL) and stirred. After dissolving solids, trichloroacetamide (1039 g) and dipotassium phosphate (111.5 g) were added to the mixture. The temperature was adjusted with heating to 25° C. and the mixture was stirred at 320 RPM for 90 minutes. 30% hydrogen peroxide (1452 g) was added over a ten minute period.

The reaction mixture was allowed to come to 20° C. and stirred at that temperature for 6 hrs., at which point conversion was checked by HPLC. Remaining enester was determined to be less than 1% by weight.

The reaction mixture was added to water (100 mL), the phases were allowed to separate, and the methylene chloride layer was removed. Sodium hydroxide (0.5 N; 50 mL) was added to the methylene chloride layer. After 20 min. the phases were allowed to separate and HCl (0.5 N; 50 mL) was added to the methylene chloride layer after which the phases were allowed to separate and the organic phase was washed with saturated brine (50 mL). The methylene chloride layer was dried over anhydrous magnesium sulfate and the solvent removed. A white solid (5.7 g) was obtained. The aqueous sodium hydroxide layer was acidified and extracted and the extract worked up to yield an additional 0.2 g of product. Yield of epoxymexrenone was 90.2%.

### EXAMPLE 2

A reactor was charged with crude Δ^{9,11}-eplerenone precursor (1628 g) and methylene chloride (6890 mL). The mixture was stirred to dissolve solids, then dipotassium phosphate (111.5 g) and trichloroacetamide (1039 g) were charged through the hatch. The temperature and agitation were adjusted to 25° C. and 320 RPM, respectively. The mixture was stirred for 90 minutes; then 30% hydrogen peroxide (1452 g) was added over a 10-15 minute period. Stirring was continued at 29-31° C. until less than 4% of the initial charge of the Δ^{9,11}-eplerenone precursor remained as determined by periodic HPLC evaluation. This required about 8 hours. At the end of the reaction, water (2400 mL) was added and the methylene chloride portion separated. The methylene chloride layer was washed with a solution of sodium sulfate (72.6 g) in water (1140 mL). After a negative test for peroxide with potassium iodide paper, the methylene chloride fraction was stirred with a caustic solution prepared from 50% sodium hydroxide (256 g) diluted in water (2570 mL) for about 45 minutes in order to remove unreacted trichloroacetamide. The methylene chloride fraction was washed sequentially with water (2700 mL), then with a solution of sodium bisulfite (190 g) in water (3060 mL).

The methylene chloride solution of eplerenone was distilled at atmospheric pressure to a final volume of approximately 2500 mL. Methyl ethyl ketone (5000 mL) was charged. The mixture was placed under vacuum distillation and solvent removed to a final volume of approximately 2500 mL. Ethanol (18.0 L) was charged and approximately 3500 mL was removed via atmospheric distillation. The mixture was cooled to 20° C. over a 3-hour period, and then stirred for 4 hours. The solid was collected on a filter and washed twice with 1170 mL of ethanol each time. The solid was dried on the filter under nitrogen for at least 30 minutes. Finally, the solid was dried in a vacuum oven at 75° C. to <5.0% limit of detection (LOD). Thus, 1100 g of the semipure eplerenone was obtained.

Recrystallization of semipure eplerenone from 8-volumes of methyl ethyl ketone (based on contained) provides pure eplerenone with a recovery of about 82%.

### EXAMPLE 3

Δ^{9,11}-eplerenone precursor (160 g crude) was combined with trichloroacetamide (96.1 g), dipotassium phosphate (6.9 g) and methylene chloride (1004 mL or 6.4 ml/g).

Water (25.6 mL) was added to the methylene chloride mixture. The quantity was adjusted to accommodate the concentration of hydrogen peroxide introduced in the following operation. In this case the water was sufficient to dilute the concentration of the subsequently added aqueous hydrogen peroxide (35 wt. %) to a desired level of 30 wt. %.

The mixture of water, steroid substrate, trichloroacetamide and dipotassium phosphate was stirred at 400 RPM and adjusted to 25° C. over a 30 to 45 minute period with a heating mantel connected to a temperature controller.

Thereafter, 35 wt. % hydrogen peroxide (138.4 mL) was added in less than 5 minutes. Although this example utilized 35% hydrogen peroxide, higher concentrations, e.g., 50 wt. %, can be used. As noted, the introduction of aqueous hydrogen peroxide having a strength greater than is desired for the reaction necessitates adding water, typically in the previous step, in order to maintain the desired concentration for the start of the reaction.

The temperature was maintained at 28 to 31° C. throughout the reaction.

The organic portion of the reaction mass was periodically sampled in order to monitor the conversion via HPLC evaluation at 240 nm. A plot of the rate of disappearance of Δ^{9,11}-eplerenone precursor vs. time gave a straight line trend with R²=0.996. The trend predicted a 98% conversion at 712 minutes. The reaction was targeted for a 95 to 98% conversion. Although the reaction was monitored at 240 nm, not all of the impurities were observed at this wavelength. In order to get a true profile of the reaction and impurities the assay was re-run at 210 nm.

Water (392 mL) was added to the mixture after 660 minutes (97.7% conversion). In the preparation of this example, the total amount of water was chosen so as to equal the volume of other water charges later in the workup. Addition of water reduced the strength of the peroxide and diminished reactivity towards the steroid components. However, the potential for the generation of low levels of oxygen was still present. The layers were allowed to separate and the lower methylene chloride layer removed (aqueous pH=6.5-7.0). Typically the hydrogen peroxide assayed at about 5 to 6% by weight. This level of concentration correlated with the consumption of 1.5 moles peroxide per mole of Δ^{9,11}-eplerenone precursor converted and a 30% starting concentration.

In a preferred mode of operation, the waste peroxide solution is disposed of via a sulfite quench. This operation is very exothermic and is preferably carried out with slow, controlled combination of the components (either forward or reverse quench modes can be used) in order to control the exotherm. The hydrogen peroxide is reduced to water while the sulfite is oxidized to sulfate during this procedure. After the sulfite quench, the quenched aqueous phase is subjected to a stream stripping operation in order to remove entrained methylene chloride. Prior to steam stripping, the aqueous phase is heated to decarboxylate the trichloroacetate salt that is produced as a by-product arising from conversion of the trichloroacetamide during the course of the epoxidation reaction. Decarboxylation prior to steam stripping prevents the trichloroacetate from reacting with methylene chloride during the stripping operation, which can otherwise result in the formation of chloroform. Decarboxylation can be effected, for example, by heating the aqueous phase at 100° C. for a time sufficient to substantially eliminate the trichoroacetate salt.

The organic phase of the reaction mixture, comprising a methylene chloride solution of eplerenone, was washed for about 15 minutes at 25° C. with an aqueous solution containing Na₂SO₃ (7.4 g) and water (122.4 mL) (pH 7-8). A negative starch iodide test (no purple color with KI paper) was observed in the organic phase at the end of the stir period. If a positive test was observed, the treatment would be repeated.

The methylene chloride fraction was washed with a dilute aqueous sodium hydroxide solution prepared from pellets (7.88 g) and water (392 mL). The mixture was stirred for 35 minutes at 25° C. and then the layers separated (aqueous pH=13). With this short contact time the trichloroacetamide is not completely hydrolyzed but is removed as the salt. In this regard, at least 2 hours is typically required to hydrolyze the trichloroacetamide to the corresponding acid salt, with release of ammonia.

The methylene chloride portion was further washed with water (392 mL). This was intended as a backup wash in case the basic interface was missed. Since the trichloroacetamide is not completely hydrolyzed during the 30-minute contact time, there is a potential for partitioning back into the organic phase once the pH is adjusted (aqueous pH=10).

The methylene chloride portion was washed with a solution of concentrated hydrochloric acid (4.1 mL) in water (352 mL) (pH 1) for about 45 minutes. At the end of this time the pH was adjusted toward neutral with the addition of a solution prepared from sodium sulfite (12.4 g) and water (40 mL) (pH 6-7).

The methylene chloride solution was concentrated via atmospheric distillation to approximate a vessel minimum stir volume (˜240 mL). About 1024 mL of methylene chloride distillate was collected. Because the preparation of this example was a “virgin run,” i.e., there was no recrystallization mother liquor available for recycle, fresh MEK (1000 mL) was added to the methylene chloride solution of eplerenone, in a proportion (1546 mL in this case) intended to mimic the recycle of mother liquor. Again, the solvent was removed via atmospheric distillation to approximate a minimum stir volume (˜240 mL). Alternatively, these distillations could have been done under vacuum.

Ethanol (2440 mL) was added to the residue. The ethanol charge correlated with 15 mL/g of estimated contained eplerenone for a crude product combined with a typical volume of MEK recrystallization mother liquor (162.7 g). No distinction was made for a virgin batch (144.8 g). Consequently, the virgin run in a campaign as operated at slightly higher volume ratios than runs that contained MEK ML for recovery.

Ethanol was distilled from the slurry (a homogeneous solution was not obtained in this treatment) at atmospheric pressure until 488 mL was removed. The quantity of ethanol removed adjusted the isolation ratio to 12 volumes (not counting the minimum stir volume of about 1.5 mL/g) times the estimated quantity of compound eplerenone contained in the crude product. Since no distinction was made for a virgin run, the isolation volume for this run was slightly inflated. The final mixture was maintained at atmospheric reflux for about one hour.

The temperature of the mixture in the distillation pot was lowered to 15° C. and, after stirring for 4 hours at this temperature, the solid was filtered. The transfer was completed with an ethanol rinse. In general, a 1-2 volume quantity based on contained eplerenone (155 to 310 mL) was utilized in production runs.

The solid was dried in a vacuum oven at 45° C. and semipure material (150.8 g) with an 89.2% assay was obtained as the output of a virgin run (154.6 g assay adjusted is the expected output for runs that include an MEK recrystallization mother liquor recovery). Generally, 94-95% of the available eplerenone was recovered after this first stage upgrade of crude product. The designated level of drying allowed isolation of the semipure eplerenone as the ethanol solvate. In this regard, the solvate does not easily release ethanol until the temperature reaches about 90° C. The solvate is preferred for further processing since the desolvated material tends to clump upon mixing with MEK in the next operation.

The solid is combined with of 2-butanone (MEK) (2164 mL). This quantity of MEK corresponds with a volume ratio of 14 mL/g vs. the estimate of contained eplerenone (includes MEK mother liquor portion).

A hot filtration of the eplerenone in MEK solution is preferably carried out prior to recrystallization, but was not employed in the laboratory run. The filtration is normally followed with a rinse quantity correlating with 2 volumes of MEK based on contained eplerenone, e.g., 310 mL. This gives a total MEK volume of 2474 mL that correlates with 16 mL/g. The hot filtration should not be operated below a ratio of 12 mL/g since this is the estimated saturation level for eplerenone in MEK at 80° C.

MEK was distilled from the solution at atmospheric pressure until 1237 mL was removed. This correlated with 8 volumes and adjusted the crystallization ratio to a volume of 8 mL/g vs. the quantity of eplerenone estimated in the semipure product. The actual volume remaining in the reactor is 8 mL/g plus the solid void estimated at 1-1.5 volumes for a total isolation target volume of 9-9.5 mL/g.

The solution (the mixture is supersaturated at this point and nucleation may occur before the cool down starts) is cooled according to the following schedule. This stepwise strategy has consistently generated polymorph II.

Cool to 65° C. and hold for 1 hour.

Cool to 50° C. and hold for 1.5 hours.

Cool to 35° C. and hold for 1 hour

Cool to 15° C. and hold for 1 hour,

Then the solid is filtered and rinsed with MEK (310 mL).

The solid was initially dried on the filter at 25° C. overnight. Then drying and desolvation were completed in a vacuum oven at 80-90° C. for ca. 4 hours. The expected dry solid weight is 119.7 g for a virgin run and 134.5 g for a run with MEK mother liquor inclusion. The LOD of the final product should be <0.1%. The filtrate (1546 mL) contained ca. 17.9 g of eplerenone. This correlated with 11.5 wt. % of adjusted input of Δ^{9,11}-eplerenone precursor. The mother liquor was saved for recovery via combination with a subsequent ethanol treatment. Data have indicated that the product eplerenone was stable up to 63 days in MEK at 40° C.

The overall assay adjusted weight yield was 76.9%. This overall yield is composed of 93, 95 and 87 assay adjusted weight % yields for the reaction, ethanol upgrade and MEK recrystallization, respectively. There is a potential 1 to 2% yield loss related to the NaOH treatment and associated aqueous washes. Inclusion of the MEK mother liquor in subsequent runs is expected to increase the overall yield by 9.5% (11.5×0.95×0.87) for an adjusted total of 86.4%.

The MEK mother liquor can be combined with a methylene chloride solution from the next epoxidation reaction and the procedure, as described above, repeated.

In view of the above, it will be seen that the several objects of the invention are achieved and other advantageous results attained. As various changes can be made in the above processes and compositions without departing from the scope of the invention, it is intended that all matter contained in the above description shall be interpreted as illustrative and not in a limiting sense.

1. A process for the preparation of an epoxy steroid compound comprising:
contacting a steroid substrate comprising olefinic unsaturation in the steroid nucleus with a peroxide compound in an epoxidation reaction zone in the presence of a peroxide activator, said peroxide compound and said steroid substrate being introduced into said reaction zone in a ratio from about one to about 7 moles peroxide compound per mole substrate; and
reacting said peroxide compound with said substrate in said reaction zone to produce a reaction mixture comprising an epoxy steroid.

2. A process as set forth in claim 1 wherein said reaction zone comprises a two phase liquid reaction medium comprising an aqueous phase comprising said peroxide compound and an organic phase comprising an organic solvent and said steroid substrate.

3. A process as set forth in claim 2 wherein said solvent is substantially immiscible with water.

4. A process as set forth in claim 3 wherein the solubility of water in said solvent is less than about 1% by weight at 25° C.

5-7. (canceled)

8. A process as set forth in claim 1 wherein said peroxide compound and said substrate are introduced into said reaction zone in a ratio between about 2 and about 7 moles peroxide per mole substrate.

9. A process as set forth in claim 8 wherein said peroxide compound and said substrate are introduced into said reaction zone in a ratio between about 2 and about 6 moles peroxide per mole substrate.

10. A process as set forth in claim 9 wherein said peroxide compound and said substrate are introduced into said reaction zone in a ratio between about 3 and about 5 moles peroxide per mole substrate.

11. A process as set forth in claim 1 wherein the epoxidation reaction is carried out to only partial conversion of said substrate to an epoxy steroid, unreacted unsaturated steroid substrate is separated from the epoxy steroid product, and the separated steroid substrate is recycled for further conversion to epoxy steroid product.

12. A process as set forth in claim 1 wherein said substrate comprises a Δ^{9,11} steroid substrate and said epoxy steroid comprises a 9,11-epoxy steroid.

13. A process as set forth in claim 12 wherein said peroxide compound and said substrate are introduced into said reaction zone in a ratio between about 2 and about 7 moles peroxide per mole substrate.

14. A process as set forth in claim 13 wherein said peroxide compound and said substrate are introduced into said reaction zone in a ratio between about 2 and about 6 moles peroxide per mole substrate.

15. A process as set forth in claim 13 wherein said peroxide compound and said substrate are introduced into said reaction zone in a ratio between about 3 and about 5 moles peroxide per mole substrate.

16. A process as set forth in claim 12 wherein said epoxidation reaction zone comprises a liquid reaction medium, said liquid reaction medium comprising a substantially water-immiscible organic solvent containing said steroid substrate.

17. A process as set forth in claim 16 wherein said liquid reaction medium further comprises an aqueous phase containing said peroxide compound.

18. (canceled)

19. A process as set forth in claim 17 wherein said steroid and solvent are introduced into a reaction vessel comprising said reaction zone, and an aqueous solution of said peroxide compound is introduced into the reaction vessel and mixed with a solution of said steroid in said solvent;
the process comprising:
introducing said solvent, said substrate, said activator into said reaction vessel; and
thereafter introducing an aqueous solution of said peroxide compound into said reaction vessel.

20. A process as set forth in claim 17 wherein said reaction medium further comprises a buffer.

21. A process as set forth in claim 17 wherein said liquid reaction medium contains said substrate and said peroxide compound in such absolute and relative proportions, and epoxidation is initiated at such temperature, that the decomposition of the peroxide content of said reaction medium in excess of that stoichiometrically equivalent to the substrate does not produce an exotherm effective to cause an uncontrolled autocatalytic decomposition of peroxide compound.

22. A process as set forth in claim 21 wherein said liquid reaction medium contains said substrate and said peroxide compound in such absolute and relative proportions, and epoxidation is initiated at such temperature, that the decomposition of the entire peroxide content of the reaction medium does not produce an exotherm effective to cause an uncontrolled autocatalytic decomposition of peroxide compound.

23. A process as set forth in claim 21 wherein such proportions and initial temperature are such that decomposition of the entire peroxide content of the reaction medium cannot produce an exotherm effective to cause an uncontrolled autocatalytic decomposition of peroxide compound.

24. A process as set forth in claim 23 wherein adiabatic decomposition of the entire peroxide content of the reaction medium does not produce an exotherm effective to cause an uncontrolled autocatalytic decomposition of peroxide compound.

25. A process as set forth in claim 17 wherein said peroxide compound comprises hydrogen peroxide.

26. A process as set forth in claim 25 wherein the concentration of hydrogen peroxide in the aqueous phase at the start of reaction between hydrogen peroxide and said substrate is at least about 25 wt. %.

27-28. (canceled)

29. A process as set forth in claim 28 wherein the concentration of said substrate in the organic phase at the start of the epoxidation reaction is between about 3 and 25 wt. %.

30. (canceled)

31. A process as set forth in any of claim 17 wherein the epoxidation reaction is conducted at a temperature not greater than about 50° C.

32-33. (canceled)

34. A process as set forth in claim 17 wherein reaction proceeds in said reaction medium to produce a two phase reaction mixture comprising an organic phase containing said 9,11-epoxy steroid and an aqueous phase containing unreacted peroxide compound.

35. A process as set forth in claim 34 wherein said aqueous phase is quenched, quenching comprising reducing peroxide contained in the aqueous phase by contact with a reducing agent.

36. A process as set forth in claim 35 wherein the aqueous phase of said liquid reaction mixture is separated from the organic phase thereof.

37. A process as set forth in claim 36 wherein said aqueous phase of said reaction mixture is separated from said organic phase prior to reduction of said peroxide compound in said aqueous phase.

38. A process as set forth in claim 37 wherein an aqueous solution of a reducing agent is added to said aqueous phase of said reaction mixture.

39. A process as set forth in claim 36 wherein said aqueous phase of said reaction mixture is added to a quenching solution, said quenching solution comprising an aqueous solution containing said reducing agent.

40. A process as set forth in claim 35 wherein said reducing agent is selected from the group consisting of alkali metal sulfite, alkali metal bisulfite, alkali metal metabisulfite, and sulfur dioxide.

41-42. (canceled)

43. A process as set forth in any of claim 35 wherein said aqueous phase of said reaction mixture is diluted with water prior to contacting said peroxide compound with said reducing agent.

44. A process as set forth in any of claim 35 wherein the aqueous phase of said reaction mixture is separated from the organic phase prior to contacting said aqueous phase with said reducing agent.

45. A process as set forth in claim 44 wherein the separated organic phase of said reaction mixture is washed with an aqueous wash liquid for removal of residual peroxide therefrom.

46. A process as set forth in claim 45 wherein said aqueous wash liquid contains a reducing agent.

47-48. (canceled)

49. A process as set forth in claim 44 wherein said quenched aqueous phase is stripped for removal of residual organic solvent therefrom.

50. A process as set forth in claim 49 wherein said activator comprises a perhaloacetamide compound, and the quenched aqueous phase is heated to decarboxylate residual perhalocarboxylate by-product prior to stripping of residual solvent.

51-54. (canceled)

55. A process as set forth in any of claim 17 wherein said solvent is selected from the group consisting of methylene chloride, dichloroethane, and methyl t-butyl ether.

56. A process as set forth in claim 12 wherein said steroid substrate corresponds to the formula: 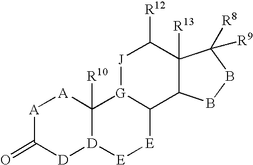
wherein
R¹⁰, R¹², and R¹³ are independently selected from the group consisting of hydrogen, halo, hydroxy, lower alkyl, lower alkoxy, hydroxyalkyl, alkoxyalkyl, hydroxycarbonyl, cyano, and aryloxy;
-A-A- represents the group —CHR¹—CHR²— or —CR¹═CR²—;
where R¹ and R² are independently selected from the group consisting of hydrogen, halo, hydroxy, alkyl, alkoxy, acyl, hydroxyalkyl, alkoxyalkyl, hydroxycarbonyl, alkoxycarbonyl, cyano, and aryloxy, or R¹ and R² together with the carbons of the steroid backbone to which they are attached form a cycloalkyl group;
-B-B- represents the group —CHR¹⁵—CHR¹⁶—, —CR¹⁵═CR¹⁶ or an α- or β-oriented group: 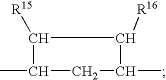
where R¹⁵ and R¹⁶ are independently selected from the group consisting of hydrogen, halo, alkyl, alkoxy, acyl, hydroxyalkyl, alkoxyalkyl, hydroxycarbonyl, alkoxycarbonyl, acyloxyalkyl, cyano, and aryloxy or R¹⁵ and R¹⁶, together with the C-15 and C-16 carbons of the steroid nucleus to which they are attached, form a cycloalkylene group, (e.g., cyclopropylene).
R⁸ and R⁹ are independently selected from the group consisting of hydrogen, alkyl, alkynyl, hydroxy, halo, lower alkoxy, acyl, hydroxyalkyl, alkoxyalkyl, hydroxycarbonylalkyl, alkoxycarbonylalkyl, acyloxyalkyl, cyano and aryloxy, or R⁸ and R⁹ together comprise a carbocyclic or heterocyclic ring structure, or R⁸ and R⁹ together with R¹⁵ or R¹⁶ comprise a carbocyclic or heterocyclic ring structure fused to the pentacyclic D ring;
-G-J- represents the group 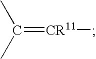
where R¹¹ is selected from the group consisting of hydrogen, alkyl, substituted alkyl or aryl;
-D-D- represents the group: 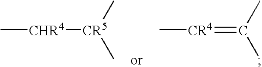
where R⁴ and R⁵ are independently selected from the group consisting of hydrogen, halo, alkyl, alkoxy, acyl, hydroxyalkyl, alkoxyalkyl, hydroxycarbonyl, alkoxycarbonyl, acyloxyalkyl, cyano and aryloxy or R⁴ and R⁵ together with the carbons of the steroid backbone to which they are attached form a cycloalkyl group;
-E-E- represents the group —CHR⁶—CHR⁷— or —CR⁶═CR⁷—;
where R⁶ is selected from the group consisting of hydrogen, halo, alkyl, alkoxy, acyl, hydroxyalkyl, alkoxyalkyl, hydroxycarbonyl, alkoxycarbonyl, acyloxyalkyl, cyano and aryloxy; and
R⁷ is selected from the group consisting of hydrogen, hydroxy, protected hydroxy, halo, alkyl, cycloalkyl, alkoxy, acyl, hydroxyalkyl, alkoxyalkyl, hydroxycarbonyl, alkoxycarbonyl, acyloxyalkyl, cyano, aryloxy, heteroaryl, heterocyclyl, acetylthio, furyl and substituted furyl, or
R⁶ and R⁷, together with the C-6 and C-7 carbons of the steroidal nucleus to which R⁶ and R⁷ are respectively attached, form a cycloalkylene group,
or R⁵ and R⁷, together with the C-5, C-6 and C-7 carbons of the steroid nucleus form a pentacyclic ring fused to the steroid nucleus and corresponding to the structure: 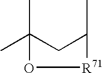
wherein R⁷¹ comprises ═CH(OH), ═CH(OR⁷²) or ═CH═O.

57. A process as set forth in claim 56 wherein said steroid substrate corresponds to the formula: 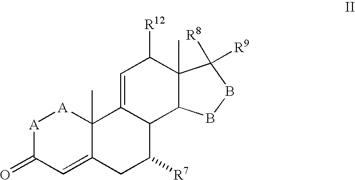
wherein
-A-A- represents the group —CHR¹—CHR²— or —CR¹═CR²—;
R¹² is selected from the group consisting of hydrogen, halo, hydroxy, lower alkyl, lower alkoxy, hydroxyalkyl, alkoxyalkyl, hydroxy carbonyl, cyano and aryloxy;
R⁷ represents an alpha-oriented lower alkoxycarbonyl or hydroxycarbonyl radical;
-B-B- represents the group —CHR⁶—CHR⁷— or an alpha- or beta- oriented group: 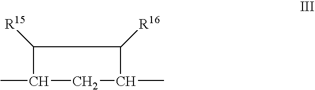
where R¹⁵ and R¹⁶ are independently selected from the group consisting of hydrogen, halo, lower alkoxy, acyl, hydroxyalkyl, alkoxyalkyl, hydroxycarbonyl, alkyl, alkoxycarbonyl, acyloxyalkyl, cyano and aryloxy; and
R⁸ and R⁹ are independently selected from the group consisting of hydrogen, alkyl, alkynyl, hydroxy, halo, lower alkoxy, acyl, hydroxyalkyl, alkoxyalkyl, hydroxycarbonyl, alkyl, alkoxycarbonyl, acyloxyalkyl, cyano and aryloxy, or R⁸ and R⁹ together comprise a carbocyclic or heterocyclic ring structure, or R⁸ or R⁹ together with R⁶ or R⁷ comprise a carbocyclic or heterocyclic ring structure fused to the pentacyclic D ring.

58. A process as set forth in claim 56 wherein R⁸ and R⁹, together with the C-17 carbon to which they are attached form a 17-spirobutyrolactone (20-spiroxane) group.

59. A process as set forth in claim 57 wherein the steroid product of said epoxidation reaction comprises eplerenone.

60. A process as set forth in claim 57 wherein said peroxide compound and said substrate are introduced into said reaction zone in a ratio between about 2 and about 7 moles peroxide per mole substrate.

61. A process as set forth in claim 60 wherein said peroxide compound and said substrate are introduced into said reaction zone in a ratio between about 2 and about 6 moles peroxide per mole substrate.

62. A process as set forth in claim 60 wherein said peroxide compound and said substrate are introduced into said reaction zone in a ratio between about 3 and about 5 moles peroxide per mole substrate.

63. A process as set forth in claim 57 wherein said epoxidation reaction zone comprises a liquid reaction medium, said liquid reaction medium comprising a substantially water-immiscible organic solvent containing said steroid substrate.

64. A process as set forth in claim 63 wherein said liquid reaction medium further comprises an aqueous phase containing said peroxide compound.

65. A process as set forth in claim 57 wherein reaction proceeds in said reaction medium to produce a two phase reaction mixture comprising an organic phase containing said 9,11-epoxy steroid and an aqueous phase containing unreacted peroxide compound.

66. A process as set forth in claim 65 wherein the organic phase of said reaction mixture is separated from the aqueous phase thereof, and said 9,11-epoxy steroid product recovered from said organic phase.

67. A process as set forth in claim 66 wherein the organic phase of said reaction mixture is washed for removal of residual peroxide compound prior to recovery of said 9,11-epoxy steroid product therefrom.

68. A process as set forth in claim 67 wherein recovery of said 9,11-epoxy steroid compound from the organic phase comprises removal of said solvent by evaporation to cause precipitation of said 9,11-epoxy steroid product.

69. A process as set forth in claim 68 wherein 9,11-epoxy steroid that has been recovered from said organic phase of said reaction mixture is contacted with a digestion solvent for removal of impurities from the precipitate by transfer to the digestion solvent phase.

70. A process as set forth in claim 69 wherein contact of said 9,11-epoxy steroid with said digestion solvent produces a slurry comprising a solvent phase containing said impurities and a solid 9,11-epoxy steroid phase.

71. A process as set forth in claim 70 wherein said solid phase comprising said 9,11-epoxy steroid is redissolved in a recrystallization solvent to produce a recrystallization solution, said recrystallization solution is heated to partially remove recrystallization solvent by evaporation, and said 9,11-steroid is crystallized from the residual recrystallization solution remaining after partial removal of said recrystallization solvent.

72. A process as set forth in claim 70 wherein the recrystallized 9,11-epoxy steroid product is separated from the recrystallization mother liquor and dried.

73. A process as set forth in claim 72 wherein said recrystallization mother liquor is recycled and combined with the 9,11-epoxy steroid compound precipitated from said organic phase of said reaction mixture; and recrystallization solvent is removed from the resulting mixture of 9,11-epoxy steroid product and mother liquor to yield a re-precipitated 9,11-epoxy steroid which is contacted with said digestion solvent.

74. A process as set forth in claim 12 wherein the epoxidation reaction is carried out to only partial conversion of said substrate to an epoxy steroid, unreacted unsaturated steroid substrate is separated from the epoxy steroid product, and the separated steroid substrate is recycled for further conversion to epoxy steroid product.

75. A process for the preparation of an epoxy steroid compound comprising:
contacting a Δ^{9,11} steroid substrate with a peroxide compound in a liquid reaction medium; and
reacting said peroxide compound with said substrate in said reaction medium to produce a reaction mixture comprising a 9,11-epoxy steroid;
said substrate and peroxide compound being contacted in such absolute and relative proportions, and at such temperature, that the decomposition of the peroxide content of said reaction medium in excess of that stoichiometrically equivalent to the substrate does not produce an exotherm effective to cause an uncontrolled autocatalytic decomposition of peroxide compound.

76-78. (canceled)

79. A process as set forth in claim 75 wherein said liquid reaction medium comprises an organic solvent containing said steroid substrate.

80. A process as set forth in claim 79 wherein said liquid reaction medium further comprises an aqueous phase containing said peroxide compound.

81. A process as set forth in claim 75 wherein the initial concentration of said peroxide compound in said aqueous phase at the start of the reaction is at least about 25 wt. %.

82-84. (canceled)

85. A process for the preparation of an epoxy steroid compound comprising:
contacting a Δ^{9,11} steroid substrate with hydrogen peroxide in a liquid reaction medium; and
reacting said substrate with hydrogen peroxide in said liquid reaction medium to produce a reaction mixture comprising a 9,11-epoxy steroid;
adding water to the reaction mixture to produce a water-diluted reaction mixture;
the composition of said water-diluted reaction mixture being such that decomposition of all the unreacted peroxide compound contained in said reaction mixture cannot produce an exotherm effective to cause an uncontrolled autocatalytic decomposition of peroxide compound.
